(19) United States
(12) Patent Application Publication
(10) Pub. No.: US 2018/0098752 A1
(43) Pub. Date: Apr. 12, 2018

(54) SYSTEMS AND METHODS FOR DETERMINING VISCOELASTIC PROPERTIES IN SOFT TISSUE USING ULTRASOUND

(71) Applicant: Duke University, Durham, NC (US)

(72) Inventors: Ned Rouze, Durham, NC (US); Kathryn Nightingale, Durham, NC (US); Mark Palmeri, Durham, NC (US)

(21) Appl. No.: 15/727,262

(22) Filed: Oct. 6, 2017

Related U.S. Application Data
(60) Provisional application No. 62/404,793, filed on Oct. 6, 2016.

(51) Int. Cl. A61B 8/08; A61B 8/00

(52) U.S. Cl. CPC A61B 8/485; A61B 8/4444; A61B 8/587; A61B 8/5223

(57) ABSTRACT

Systems and methods for determining viscoelastic properties in soft tissue using ultrasound are disclosed. According to an aspect, a method includes using an ultrasound probe to acquire soft tissue data. The method also includes determining a first group shear wave speed having a first frequency spectra. Further, the method includes determining a second group shear wave speed having a second frequency spectra. The method also includes determining one or more viscoelastic properties of the soft tissue based on the first group shear wave speed and the second group shear wave speed.

### CROSS REFERENCE TO RELATED APPLICATION

This application claims priority to U.S. Provisional Patent Application No. 62/404,793, filed on Oct. 6, 2016 and titled SYSTEMS AND METHODS FOR MEASURING STIFFNESS AND VISCOSITY IN SOFT TISSUE, the disclosure of which is incorporated herein by reference in its entirety.

### TECHNICAL FIELD

The present disclosure relates to soft tissue imaging using ultrasound. In particular, the present disclosure relates to systems and methods for measuring stiffness and viscosity in soft tissue using ultrasound.

### BACKGROUND

The mechanical properties of soft tissue are of interest to many areas of research including the study of injury mechanisms in biomechanics and the creation of realistic computer simulations of surgical procedures. In medicine, the stiffness of tissue can be related to pathology and is often used to diagnose disease. In a clinical setting, the quantification of tissue stiffness is useful for monitoring the progression of disease, and its response to treatment.

One way that tissue stiffness can be evaluated is through measurement of the group shear wave speed since, in the simplest model of tissue, the shear modulus of a material is directly related to the square of the group speed. Shear waves can be generated with an impulsive excitation, for example, as is done in acoustic radiation force impulse (ARFI) imaging or with an external vibration source. The tissue displacements in these waves can be tracked ultrasonically through time following the excitation, and the group shear wave speed and shear modulus of the tissue determined from these measurements.

For characterization of viscoelastic materials such as soft tissue, it is necessary to measure the tissue viscosity in addition to stiffness. In particular, tissue viscosity is thought to be related to steatotic (fatty) liver disease and potentially other diseases, such as hepatic inflammation. A goal of many manufacturers of commercial ultrasound scanners is to develop the capability to measure tissue viscosity to supplement existing capabilities to measure tissue stiffness.

To measure tissue viscosity, various studies have performed a Fourier decomposition of the shear wave signal and calculated the frequency dependent phase velocity, which may then be used to determine the stiffness and viscosity using a two-parameter material model. Compared to measurement of group shear wave speeds, spectral analysis methods can be very sensitive to measurement noise which leads to low measurement yields among repeated measurements and low confidence in the accuracy of model parameters determined from model dependent fitting. Despite advances for determining soft tissue properties, there is a continuing need for improved systems and techniques for determining such properties using ultrasound.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Disclosed herein are systems and methods for determining viscoelastic properties in soft tissue using ultrasound. According to an aspect, a method includes using an ultrasound probe to acquire soft tissue data. The method also includes determining a first group shear wave speed having a first frequency spectra. Further, the method includes determining a second group shear wave speed having a second frequency spectra. The method also includes determining one or more viscoelastic properties of the soft tissue based on the first group shear wave speed and the second group shear wave speed.

### BRIEF DESCRIPTION OF THE SEVERAL VIEWS OF THE DRAWINGS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting.

The foregoing aspects and other features of the present subject matter are explained in the following description, taken in connection with the accompanying drawings, wherein:

FIG. 1 is a block diagram of a system for measuring the stiffness and viscosity in viscoelastic materials according to embodiments of the present disclosure;

FIG. 2 is a flow chart depicting an example method for measuring stiffness and viscosity in viscoelastic materials according to the embodiments of the present disclosure;

FIG. 3 is a flow chart diagram depicting another example method for measuring stiffness and viscosity in viscoelastic materials according to the embodiment of the present disclosure;

FIGS. 4A-4D are visual representation images of acoustic radiation force excitation intensities;

FIGS. 5A-5H are graphical comparison results of shear wave propagation;

FIGS. 6A and 6B are graphs depicting shear wave propagation;

FIGS. 7A-7D are graphical comparison results of shear wave propagation using lookup tables according to embodiments of the present disclosure;

FIGS. 8A-8C depict comparison result graphics of shear wave propagation using lookup tables; and

FIGS. 9A and 9B show comparison result graphics of shear wave propagation using excitation configurations.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The functional units described in this specification have been labeled as devices. A device may be implemented in programmable hardware devices such as processors, digital signal processors, central processing units, field programmable gate arrays, programmable array logic, programmable logic devices, cloud processing systems, or the like. The devices may also be implemented in software for execution by various types of processors. An identified device may include executable code and may, for instance, comprise one or more physical or logical blocks of computer instructions, which may, for instance, be organized as an object, procedure, function, or other construct. Nevertheless, the executables of an identified device need not be physically located together, but may comprise disparate instructions stored in different locations which, when joined logically together, comprise the device and achieve the stated purpose of the device.

An executable code of a device may be a single instruction, or many instructions, and may even be distributed over several different code segments, among different applications, and across several memory devices. Similarly, operational data may be identified and illustrated herein within the device, and may be embodied in any suitable form and organized within any suitable type of data structure. The operational data may be collected as a single data set, or may be distributed over different locations including over different storage devices, and may exist, at least partially, as electronic signals on a system or network.

The described features, structures, or characteristics may be combined in any suitable manner in one or more embodiments. In the following description, numerous specific details are provided, to provide a thorough understanding of embodiments of the disclosed subject matter. One skilled in the relevant art will recognize, however, that the disclosed subject matter can be practiced without one or more of the specific details, or with other methods, components, materials, etc. In other instances, well-known structures, materials, or operations are not shown or described in detail to avoid obscuring aspects of the disclosed subject matter.

As referred to herein, the term “computing device” should be broadly construed. It can include any type of device including hardware, software, firmware, the like, and combinations thereof. A computing device may include one or more processors and memory or other suitable non-transitory, computer readable storage medium having computer readable program code for implementing methods in accordance with embodiments of the present disclosure. A computing device can be any suitable type of computer, for example, desktop computer, a laptop computer, a tablet computer, or the like. A computing device may also be a mobile computing device such as, for example, but not limited to, a smart phone, a cell phone, a pager, a personal digital assistant (PDA), a mobile computer with a smart phone client, or the like.

Further, as used herein, the term “memory” is generally a storage device of a computing device. A non-exhaustive list of more specific examples of the memory may include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing.

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. For example, exemplary embodiments are intended to cover all software or computer programs capable of enabling processors to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed below.

In accordance with the exemplary embodiments, the disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl, or other suitable programming languages.

As used herein, the term “frequency” may refer to a number of periodic cycles or periodic waves or signals over a given unit of time.

As used herein, the term “spectrum” or “spectra” may refer to a group of one or more frequencies.

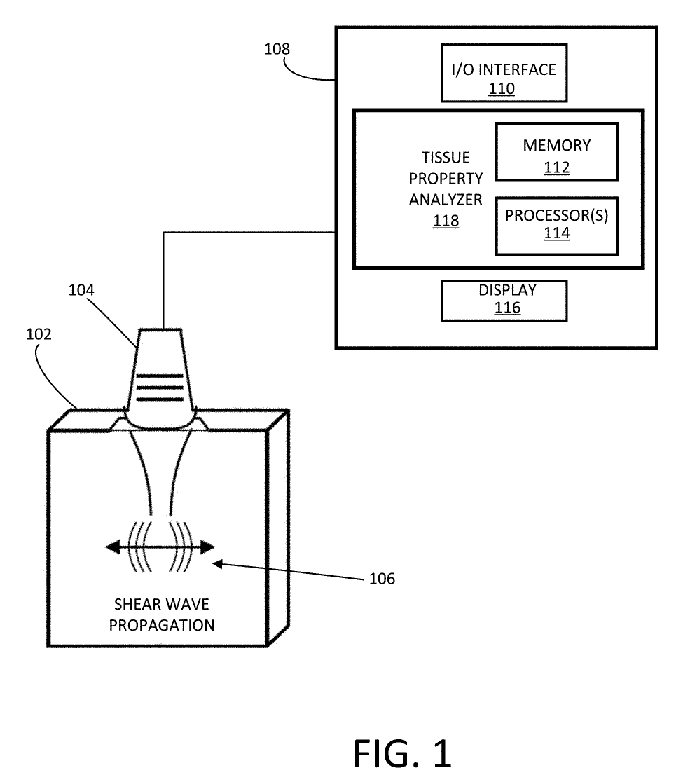

The presently disclosed subject matter is now described in more detail. For example, FIG. 1 illustrates a block diagram of an example system for determining viscoelastic properties in soft tissue in accordance with embodiments of the present disclosure. Referring to FIG. 1, the system may be used to determine viscoelastic properties of a soft tissue sample 102. The system includes an ultrasound probe 104, a computing device 108, an I/O interface 110, a memory 112, one or more processors 114, a display 116, and a tissue property analyzer 118. The computing device 108 may be any suitable computer configured to perform medical imaging including ultrasonic imaging, ultrasound radiation force imaging, acoustic radiation force imaging, or the like. The soft tissue sample 102 may be skin, fascia, tendons, ligaments, muscles, nerves, organs, blood vessels, or other types of soft tissue. As shown in FIG. 1, the ultrasound probe 104 may interact with the soft tissue sample 102 by transducing ultrasonic waves upon the soft tissue sample 102 using acoustic radiation force to excite the soft tissue sample 102 impulsively. As further shown in FIG. 1, as the ultrasonic waves are emitted throughout the soft tissue sample, shear wave propagation 106 may be observed. The group shear wave speed 106 from shear wave propagation may be measured using suitable ultrasonic tracking. The ultrasound probe 104 may transmit data signals indicating multiple group shear wave speeds to the shear wave propagating device 108.

The computing device 108 may include an I/O interface 110, a memory 112, one or more processors 114, a display 116, and a tissue property analyzer 118. The I/O interface 110 may be any suitable logic configured to interpret input received via an input device such as keyboard or touchscreen. The I/O interface 110 may assess hardware by reading and writing to specific memory locations with the memory 112. The user can use any other suitable user interface of a computing device, such as a keypad, to select the display icon or display objects. Memory 112 may be any type of memory suitable to store processor executable instructions, data, image data, or the like. The display 116 may be any suitable display configured to display, project, or otherwise present soft tissue sample images or the like in accordance with the present disclosure. The display 116 can be a touchscreen display.

A tissue property analyzer 118 may be implemented by the memory 112 and processor(s) 114. Alternatively, for example, the tissue property analyzer 118 may be implemented by any suitable hardware, software, firmware, or combinations thereof. The processor(s) 114 may be any processor or dual processor that may be configured as an array of programmable logic gates or may be configured a microprocessor in combination with memory 112 in which a program executable on the microprocessor is stored. The processor(s) 114 may include processor-executable instructions to determine a first group shear wave speed having a first frequency spectra, determine a second group shear wave speed having a second frequency spectra, wherein the second frequency spectra is different than the first frequency spectra, and determine one or more viscoelastic properties of the soft tissue based on the first group shear wave speed and the second group shear wave speed.

The tissue property analyzer 118 may control functionality of the computing device 108, its components, and the ultrasonic probe 104. For example, the tissue property analyzer 118 may control the irradiation timing of each of the ultrasonic probe 104 and may also determine a first group shear wave speed having a first frequency spectra and determine a second group shear wave speed having a second frequency spectra, wherein the second frequency spectra is different than the first frequency spectra, the process of which will be described in greater detail below and one or more viscoelastic properties of the soft tissue 102 based on the first group shear wave speed and the second group shear wave speed. The tissue property analyzer 118 may also determine a first group shear wave speed having a first frequency spectra based on tissue displacement and tissue velocity signals acquired by the ultrasound probe 104, and determine a second group shear wave speed having a second frequency spectra based on tissue displacement and tissue velocity signals acquired by the ultrasound probe 104. The second frequency spectra is different than the first frequency spectra. In determining the above features, the tissue property analyzer 118 may generate ultrasound images depicting the measured stiffness and viscosity in the viscoelastic material using the group shear wave speeds. The process of generating ultrasound images according to the disclosure of the present subject matter will now be described below.

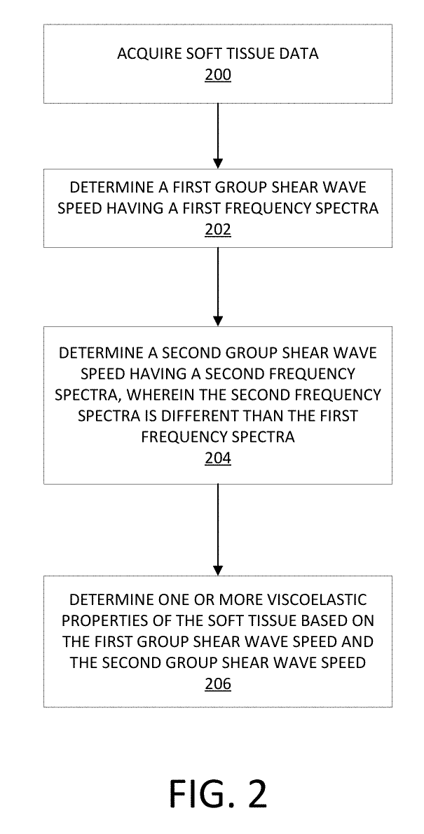

FIG. 2 illustrates a flow chart of an example method for determining viscoelastic properties in sot tissue using ultrasound in accordance with embodiments of the present disclosure. In this example, the method is described as being implemented by the system shown in FIG. 1, although it should be understood that the method may be implemented by any suitable system configured to acquire soft tissue data and to analyze such data. Referring to FIG. 2, the method includes acquiring 200 soft tissue data. For example, the ultrasound probe 104 may be operated to acquire soft tissue data of the soft tissue 102.

The method of FIG. 2 also includes determining 202 a first group shear wave speed having a first frequency spectra. The method may also include determining 204 a second group shear wave speed having a second frequency spectra. The second frequency spectra is different than the first frequency spectra. Continuing the aforementioned example, the tissue property analyzer 118 may receive the acquired soft tissue data of the soft tissue 102. Based on this acquired soft tissue data, the tissue property analyzer 118 may determine the first group shear wave speed having the first frequency spectra, and determine the second group shear wave speed having the second frequency spectra

The method of FIG. 2 also includes determining 206 one or more viscoelastic properties of the soft tissue based on the first group shear wave speed and the second group shear wave speed. Continuing the aforementioned example, the tissue property analyzer 118 may determine one or more viscoelastic properties of the soft tissue based on the first group shear wave speed and the second group shear wave speed. The tissue property analyzer 118 may also control the display 116 or another user interface to present information or graphical data indicating the viscoelastic properties of the soft tissue 102.

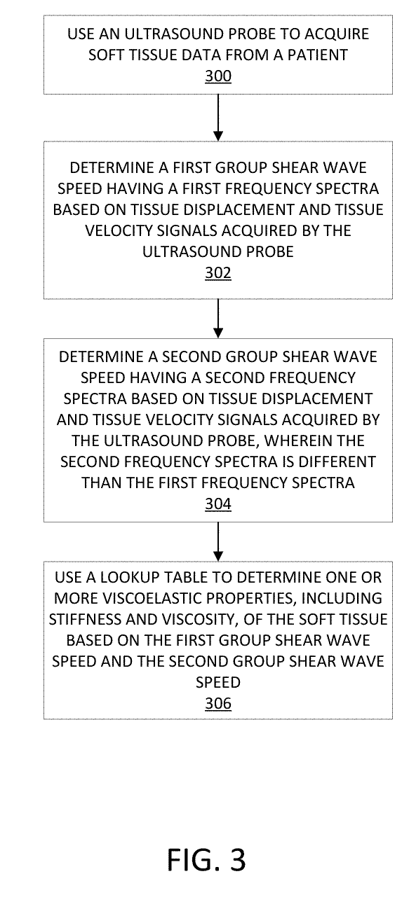

FIG. 3 illustrates a flow chart diagram of another example method for determining viscoelastic properties in sot tissue using ultrasound in accordance with embodiments of the present disclosure. In this example, the method is described as being implemented by the system shown in FIG. 1, although it should be understood that the method may be implemented by any suitable system configured to acquire soft tissue data and to analyze such data. Referring to FIG. 3, the method includes using 300 an ultrasound probe 104 to acquire soft tissue data. For example, the ultrasound probe 104 shown in FIG. 1 may be used to acquire soft tissue data of the soft tissue 102.

The method of FIG. 3 includes determining 302 a first group shear wave having a first frequency spectra based on tissue displacement and tissue velocity signals acquired by the ultrasound probe 104. Further, the method of FIG. 3 includes determining 304 a second group shear wave speed having a second frequency spectra based on tissue displacement and tissue velocity signals acquired by the ultrasound probe. The second frequency spectra is different than the first frequency spectra 304. Continuing the aforementioned example, the tissue property analyzer 118 may receive the acquired soft tissue data of the soft tissue 102. Based on this acquired soft tissue data, the tissue property analyzer 118 may determine the first group shear wave speed having the first frequency spectra, and determine the second group shear wave speed having the second frequency spectra.

The method of FIG. 3 using 306 a lookup table to determine one or more viscoelastic properties, including stiffness and viscosity, of the soft tissue based on the first group shear wave speed and the second group shear wave speed. The lookup table can be determined mathematically using at least excitation force, tracking configuration, and material properties of the soft tissue, that associates the one or more viscoelastic properties with the first group shear wave speed and the second group shear wave speed. Further details and examples of generating and using a lookup table in accordance with embodiments of the present disclosure are provided herein. Also, the tissue property analyzer 118 may also control the display 116 or another user interface to present information or graphical data indicating the viscoelastic properties of the soft tissue 102.

In embodiments, a method is disclosed for measuring the stiffness and viscosity in viscoelastic materials using group shear wave speeds calculated using both tissue displacement and tissue velocity signals. The method does not rely on the Fourier decomposition and spectral analysis methods previously used. The method also provides robust characterization of the stiffness and viscosity in viscoelastic tissues. The method may be implemented on a computing function of a system that includes suitable function, such as an ultrasound function. In other embodiments, the method may be stored as instructions on a non-transitory computer readable medium for use by or in connection with the computing function.

It is noted that while examples provided herein sometimes relate to ultrasound and ultrasonic techniques, but the subject matter disclosed herein should not be construed as so limited. The techniques of characterizing materials using group shear wave speeds also applies to measurements using other techniques, such as where excitations are performed using mechanical vibrations and/or the shear wave tracking is performed using magnetic resonance or optical techniques.

A method in accordance with the present disclosure can leverage ultrasonic technologies currently using acoustic radiation force to excite tissue impulsively and to observe shear wave propagation and measure the group shear wave speed using ultrasonic tracking. Several propagation speeds can be determined from these measurements including, for example, the propagation speed V_{disp} calculated using the tissue displacement signal u(x, t), and the propagation speed Vᵥₑₗ calculated using the tissue velocity signal v(x,t).

The stiffness μ and viscosity η of tissue can be obtained directly from measurements of two group shear wave speeds such as V_{disp} and Vᵥₑₗ by determining lookup tables μ(V_{disp}, Vᵥₑₗ) and η(V_{disp}, Vᵥₑₗ). The data in these tables are determined by solving the Navier equation of motion in the frequency domain [11] to determine an expression for the temporal Fourier transform ũ(x, ω) of the tissue displacement signal for shear wave propagation observed along the x-axis as shown in equation 1.

$$\tilde{u} ( x , ω ) = \frac{- i π}{2} \frac{\tilde{W} ( ω )}{μ + i ω η} {\sum }_{m = - ∞}^{∞} \{ {H}_{m}^{( 2 )} ( x ) {\int }_{0}^{r} {f}_{m} ( {r}_{s} ) {J}_{m} ( κ {r}_{s} ) {r}_{s} {dr}_{s} + {J}_{m} ( x ) {\int }_{r}^{∞} {f}_{m} ( {r}_{s} ) {H}_{m}^{( 2 )} ( κ {r}_{s} ) {r}_{s} {dr}_{s} \} \qquad [ 1 ]$$

\tilde{W}(ω) is the Fourier transform of the time dependence W(t) of the excitation force. Jₘ(z) and Hₘ⁽²⁾(z) are Bessel functions. κ=\sqrt{ρω²/(μ+iωη)} is the complex wavenumber. fₘ(rₛ) are the coefficients of a Fourier series expansion of the excitation force f (rₛ, θ) as shown in equation 2.

fₘ(rₛ)=∫hd 0^{2π}f(rₛ,θ)e^{-imθ}dθ [2]

The tissue displacement signal u(x, t) is calculated using the inverse Fourier transform of ũ(x, ω) as shown in equation 3.

$$u ( x , t ) = \frac{1}{2 π} {\int }_{- ∞}^{∞} \tilde{u} ( x , ω ) {e}^{i ω t} d ω \qquad [ 3 ]$$

The tissue velocity signal v(x, t) is calculated by differentiation of u(x, t) with respect to time as shown in equation 4.

$$v ( x , t ) = \frac{d}{dt} u ( x , t ) \qquad [ 4 ]$$

The group shear wave speeds V_{disp} and Vᵥₑₗ are calculated from u(x, t) and v(x, t) using exactly the same procedure, for example, time-to-peak[4] or cross-correlation[12], as used for the measurement of V_{disp} and Vᵥₑₗ from experimental data.

Values of the group shear wave speeds V_{disp} and Vᵥₑₗ are calculated using the procedure described above for the specific excitation and shear wave tracking configurations used experimentally, and for a wide range of values of tissue stiffness μ and viscosity η, to give the relations V_{disp}(μ, η) and Vᵥₑₗ (μ, η). These relations can be inverted to give the lookup tables μ(V_{disp}, Vᵥₑₗ) and η(V_{disp}, Vᵥₑₗ) that are used to determine the tissue stiffness μ and viscosity η from experimentally measured group shear wave speeds.

In another representative embodiment, other experimental measurements of group shear wave speeds may be used in addition to V_{disp} and Vᵥₑₗ. Examples of these speeds include the group shear wave speed determined using the shear wave acceleration signal, V_{acc}, and the speeds determined by analyzing the experimental signals using fractional derivative operations instead of integer derivative operations used to determine the displacement, velocity, or acceleration speeds. Any two of these speeds may be used with two dimensional lookup tables. Furthermore, different combinations of speeds may be used to check the consistency of the data and to reduce errors introduced by noise in the experimental measurements.

The determination of stiffness and viscosity of soft tissue can require two experimental measurements. Described herein is the use of group shear wave speeds determined using the shear wave displacement signal, V_{disp}, and the speed determined using the shear wave velocity signal, Vᵥₑₗ. However, these particular speeds were used as only an example as described herein. Other experimental measurements of group shear wave speeds may also be used. Examples of these speeds include the group shear wave speed determined using the shear wave acceleration signal, V_{acc}, and the speeds determined by analyzing the experimental signals using fractional derivative operations instead of integer derivative operations used to determine displacement, velocity, or acceleration speeds. Any two of these speeds may be used to check the consistency of the data and to reduce errors introduced by noise in the experimental measurements.

Previous efforts to analyze shear wave motion from these sources have been based on Graff's solution [5] of the Navier equation of motion for shear wave propagation in an elastic material for the specific case of a source with zero width and infinite extent along the z-axis that is applied harmonically in time. Graff's solution [5] gives the shear wave displacement signal u(r, t) as a function of position and time.

$$u ( r , t ) = \frac{i}{4} {H}_{0}^{( 2 )} ( kr ) {e}^{i {ω}_{0} t} , \qquad ( 3 )$$

where H0(2)(kr) is a Hankel function with wavenumber k=ρω02/μ from (1), and ω0 is the excitation frequency. Note that in (3), the Hankel function type and the sign of the phase in the factor eiω0t have been switched compared to Graff [5] to agree with the sign convention used by Rouze, et al. [8] For non-harmonic excitations, the shear wave signal can be expressed as a superposition of solutions of the form of (3) with amplitudes A(ω0),

$$u ( r , t ) = {\int }_{- ∞}^{∞} A ( {ω}_{0} ) \frac{i}{4} {H}_{0}^{( 2 )} ( kr ) {e}^{i {ω}_{0} t} d {ω}_{0} . \qquad ( 4 )$$

This analysis may also be extended to the case of a viscoelastic material by replacing the wavenumber k with a complex wavenumber k=\sqrt{ρω²/μ(ω)} from (2). For experimentally measured signals, different frequency components of the shear wave signal can be isolated by calculating the Fourier transform in time. Here, the temporal Fourier transform of a spatial-temporal signal may be represented using a tilde notation, for example,

\tilde{u}(r,ω)= [symbol] ₜ{u(r,t)}=∫₋₍ₓ₎^{(x)}u(r,t)e^{-iωt}dt. (5)

Then, with k→κ for a viscoelastic material, the temporal Fourier transform of the shear wave signal u(r, t) from (4) is given by

$$\tilde{u} ( r , ω ) = A ( ω ) \frac{i π}{2} {H}_{0}^{( 2 )} ( κ r ) . \qquad ( 6 )$$

Often, this result is expressed using the asymptotic form of the Hankel function,

$${H}_{0}^{( 2 )} ( κ r ) \sim \frac{1}{\sqrt{κ r}} {e}^{- i κ r} = \frac{1}{\sqrt{( k - i α ) r}} {e}^{- ikr} {e}^{- α r} . \qquad ( 7 )$$

Two procedures are commonly used to measure the phase velocity and attenuation from the Fourier transform signal ũ(r, ω). First, as described by Deffieux, et al.[13], the phase of the asymptotic Hankel function (7) varies linearly with the coordinate r, so a linear fit of phase vs. position can be used to measure the wavenumber k and phase velocity c(ω)=ω/k. Similarly, the attenuation α(ω) can be measured using the exponential decay with position in (7), or by using the Hankel function dependence in (6) [9]. A second method to measure the phase velocity and attenuation is by constructing the two-dimensional Fourier transform (2DFT) of the spatial-temporal shear wave signal and to measure the phase velocity and attenuation from the location and width of the 2DFT signal at each specific temporal frequency [8, 14]. It is important to realize that both of these analysis methods depend on the specific form of the solution (3), and that this solution does not account for the size and shape of an ARFI excitation which are known to give biased results for the phase velocity [8] and group shear wave speed [15].

Typically, investigations designed to characterize the properties of viscoelastic materials do not report the phase velocity c(ω) or attenuation a(ω), but instead, use a material model to parametrize these quantities in terms of a small number of material constants. One common model is a Voigt model [11, 16] which expresses the shear modulus in terms of the stiffness μ0 and viscosity η as

μ(ω)=μ₀+iωη. (8)

A second, commonly used material model is a linear dispersion model in which the phase velocity is modeled as a linear function of frequency,

$$c ( f ) = {c}_{0} + \frac{dc}{df} f . \qquad ( 9 )$$

This relation is also characterized by two parameters, for example, the phase velocity at a specific frequency and the dispersion slope dc/df. Fitting experimental data to these models can be difficult, particularly when fitting the phase velocity for the Voigt model because of the complicated frequency dependence of c(ω) that includes frequency ranges with both positive and negative curvature. In noisy data, it is easy for the fitting procedure to attempt to reproduce frequency dependent structures introduced by experimental noise which can lead to unrealistic parameters. This behavior led Nightingale, et al. [18], to analyze the phase velocity data obtained in NAFLD human liver subjects using a linear dispersion model which is less susceptible to experimental noise.

In an alternate embodiment of the present disclosure includes characterizing the viscoelastic properties of materials using measured group shear wave speeds. For instance, the group shear wave speed Vdisp may be determined using the shear wave displacement signal u(r, t) and the group shear wave speed Vvel may be determined using the particle velocity signal. For viscoelastic materials, these group speeds may not be equal because the phase velocity c(ω) increases with frequency and, in the temporal Fourier domain, the process of differentiation in (10) weights the velocity signal by a factor of iω compared to the displacement signal,

\tilde{v}(r,ω)=iω\tilde{u}(r, ω). (11)

Thus, in a viscoelastic material, when averaging over the frequency content of the shear wave, the larger phase velocities at higher frequencies will be weighted more heavily for the velocity group speed, as compared to the displacement group speed, and it may be expected that Vvel >Vdisp. However, for an elastic material, the phase velocity is independent of frequency and the increased weighting at higher frequencies will not lead to different speeds, and it may be expected that Vdisp=Vvel. The method of the present disclose is robust and improves upon the current technologiy of determining the stiffness and viscosity of a viscoelastic material. First, unlike previous methods that rely on the Fourier decomposition of a shear wave signal to isolate specific frequency components, the proposed method uses only group shear wave speeds which can be easily measured experimentally. Second, based on the discussion above, the difference ΔV=Vvel−Vdisp gives a direct measure of the material viscosity in the same way that the group shear wave speed gives a measure of the material stiffness.

In an alternate embodiment of the present disclosure, a model of shear wave propagation in a viscoelastic material is developed and describes how the group shear wave speeds Vdisp and Vvel can be calculated from the shear wave displacement signal u(\tilde{r},t) and particle velocity signal v(\tilde{r},t) for a specific shear modulus μ(ω). Specifically, the equation of motion is solved for the shear wave displacement signal u(\tilde{r},t) generated by a localized, impulsive excitation force. The calculation of the group shear wave speeds Vdisp and Vvel from this signal is complicated for two reasons. First, time-to-peak (TTP) and cross-correlation (XCORR) estimators are commonly used to determine the shear wave arrival time as a function of position; however, these estimators yield different expressions for the arrival times and group speeds Vdisp and Vvel in dispersive materials. Second, it is not possible to obtain a closed form expression for Vdisp and Vvel that accounts for the specific range of positions considered in the linear regression of arrival time vs position. Instead, the group speeds are determined by analyzing the calculated shear wave signals using exactly the same procedure as used for the analysis of experimentally measured signals. The method presented here is expected to be robust compared to previous methods used to determine the stiffness and viscosity of a viscoelastic material. First, unlike previous methods that rely on the Fourier decomposition of a shear wave signal to isolate specific frequency components, the proposed method uses only group shear wave speeds which can be easily measured experimentally. And second, based on the discussion in the previous paragraph, it is shown that the difference ×V=Vvel−Vdisp gives a direct measure of the material viscosity in the same way that the group shear wave speed gives a measure of the material stiffness.

### A. ARFI Excitation

Cylindrical coordinates r, θ, and z are used to describe the ARFI excitation force and shear wave propagation. The excitation force \tilde{f}(\tilde{r},t) is assumed to be directed along the z axis with negligible z dependence so that it can be written as

\vec{f}(\vec{r},t)=f(r,θ)W(t)\tilde{z} (12)

where the window function W (t) gives time dependence of the excitation. For asymmetric sources, the r and θ dependence in (12) can be separated by expanding f(r, θ) in a Fourier series of the form

$$f ( r , θ ) = {\sum }_{l = - ∞}^{∞} {f}_{l} ( r ) {e}^{il θ} \qquad ( 13 )$$

where the expansion coefficients fi(r) are given by

$${f}_{l} ( r ) = \frac{1}{2 π} {\int }_{0}^{2 π} f ( r , θ ) {e}^{- il θ} d θ . \qquad ( 14 )$$

In Section C, the assumption may be made that the excitation force is localized so that f(r, θ) can be neglected for radial coordinates outside a maximum source radius R.

### B. Equation of Motion

An analytic description of shear wave propagation can be found by solving the Navier equation of motion for the shear wave displacement signal u(\vec{r},t) [5]. Because the shear modulus for a viscoelastic material is a complex, frequency dependent quantity, it is convenient to work in the temporal Fourier domain so that the equation of motion for the ith component of the tissue displacement is given by [8]

μ(ω)ũ_{i,jj}(r,θ,ω)+fᵢ(r,θ,ω)\tilde{W}(ω)=−ρω²ũᵢ(r,θ,ω). (15)

Because the excitation force (12) has only a nonzero \hat{z} component, the solution of (15) will only involve the z component of the displacement. Thus, the component subscript i was dropped in the following with the understanding that the displacement signal ũ(r,θ,ω) refers to the \hat{z} component.

As with the excitation force (13), the radial and angular dependence in the displacement signal ũ(r,θ,ω) can be separated using a Fourier series

$$\tilde{u} ( r , θ , ω ) = {\sum }_{m = - ∞}^{∞} {\tilde{u}}_{m} ( r , ω ) {e}^{im θ} . \qquad ( 16 )$$

The Fourier coefficients ũₘ(r,ω) of the solution can be found by inserting (16) and (13) into (15) and using the orthogonality of the exponential factors so that 1=m. In addition, in cylindrical coordinates, the Laplacian operator ũ_{,jj}=∇²ũ is written in terms of partial derivatives with respect to the radial coordinate r and angular coordinate θ so that

$$μ ( ω ) [ \frac{{\partial }^{2}}{\partial {r}^{2}} + \frac{1}{r} \frac{\partial }{\partial r} - \frac{{m}^{2}}{{r}^{2}} ] {\tilde{u}}_{m} ( r , ω ) + {f}_{m} ( r ) \tilde{W} ( ω ) = - ρ {ω}^{2} {\tilde{u}}_{m} ( r , ω ) . \qquad ( 17 )$$

Equation (17) can be solved by applying the Hankel transform [19] of order m, rearranging and applying the inverse transform,

$$\begin{matrix}{\tilde{u}}_{m} ( r , ω ) = \frac{\tilde{W} ( ω )}{μ ( ω )} {m}_{- 1}^{} \{ \frac{{m}_{} \{ {f}_{m} ( {r}_{s} ) \}}{{ξ}^{2} - {κ}^{2}} \} \\ = \frac{\tilde{W} ( ω )}{μ ( ω )} {\int }_{0}^{∞} \frac{1}{{ξ}^{2} - {κ}^{2}} [ {\int }_{0}^{∞} {f}_{m} ( {r}_{s} ) {J}_{m} ( ξ {r}_{s} ) {r}_{s} {dr}_{s} ] {J}_{m} ( ξ r ) ξ d ξ\end{matrix} \qquad ( 18 )$$

where Jₘ(z) is a Bessel function of the first kind of order m, ξ is the Hankel transform variable, and κ=κ(ω)=\sqrt{ρω²/μ(ω)} is the complex wavenumber from (2). Note that in (18), the integral over the source term is written in terms of a dummy integration coordinate rₛ to distinguish it from the coordinate r where the shear wave is observed. The ξ integral in (18) can be evaluated analytically using 6.541.1 of [20] so that

$${\tilde{u}}_{m} ( r , ω ) = \frac{\tilde{W} ( ω )}{μ ( ω )} \{ {K}_{m} ( i κ r ) {\int }_{0}^{r} {f}_{m} ( {r}_{s} ) {I}_{m} ( i κ {r}_{s} ) {r}_{s} {dr}_{s} + {I}_{m} ( i κ r ) {\int }_{r}^{∞} {f}_{m} ( {r}_{s} ) {K}_{m} ( i κ {r}_{s} ) {r}_{s} {dr}_{s} \} \qquad ( 19 )$$

where Iₘ(z) and Kₘ(z) are modified Bessel functions.

### C. Localized Source

Typically, acoustic radiation force sources are localized so that the excitation force f(rs, θ) is negligible for positions outside a maximum source radius R. Then, for positions outside the source with r>R, the second term in braces in (19) can be neglected so that ũₘ(r,ω) is given by

$${\tilde{u}}_{m} ( r , ω ) = \frac{\tilde{W} ( ω )}{μ ( ω )} {S}_{m} ( ω ) {K}_{m} ( i κ r ) \qquad ( 20 )$$

where the source integral Sₘ(ω) given by

Sₘ(ω)=∫₀^{B}fₘ(rₛ)Iₘ(iκrₛ)rₛdr_{s.} (21)

In the remainder of this paper, we only consider shear wave signals observed at positions outside a localized source and use (20) for ũₘ(r,ω).
D. Shear wave signals and group speeds

A complete expression for the shear wave displacement signal u(r,74 ,t) can be found by combining expressions (16) and (20) and calculating the inverse Fourier transform in time,

$$u ( r , θ , t ) = \frac{1}{2 π} {\sum }_{m = - ∞}^{∞} {e}^{im θ} {\int }_{- ∞}^{∞} \frac{\tilde{W} ( ω )}{μ ( ω )} {S}_{m} ( ω ) {K}_{m} ( i κ r ) {e}^{i ω t} d ω . \qquad ( 22 )$$

The shear wave velocity signal v(r,θ,t) can be calculated by differentiation of u(r,θ,t) with respect to time as in (10), or equivalently, using (11) and in the factor iω in (22),

$$v ( r , θ , t ) = \frac{1}{2 π} {\sum }_{m = - ∞}^{∞} {e}^{im θ} {\int }_{- ∞}^{∞} \frac{\tilde{W} ( ω )}{μ ( ω )} {S}_{m} ( ω ) {K}_{m} ( i κ r ) {e}^{i ω t} i ω d ω \qquad ( 23 )$$

In particular, we note that the size and shape of the excitation force are included in expressions (22) and (23) through the Fourier expansion of the excitation force and the source integrals Sₘ(ω). Similarly, the effect on the time dependence W(t) of the excitation force on the shear wave signals is accounted for in the factor of \tilde{W}(ω).

In most experiments, the shear wave group speed is found by observing the shear wave signal for propagation in a direction perpendicular to the excitation axis, measuring the wave arrival time at a series of positions r outside the source, and performing a linear regression of the arrival time vs. position. TTP or XCORR estimators are commonly used to determine the wave arrival time. For TTP measurements, an expression for the arrival time tₚₖ of the peak displacement signal can be found by differentiating (22) with respect to time and setting the result to zero,

$$0 = {\sum }_{m = - ∞}^{∞} {e}^{im θ} {\int }_{- ∞}^{∞} \frac{\tilde{W} ( ω )}{μ ( ω )} {S}_{m} ( ω ) {K}_{m} ( i κ r ) {e}^{i ω {t}_{pk}} ω d ω . \qquad ( 24 )$$

The group speed Vdisp is found by assuming the arrival time is a linear function of position and performing a linear regression using the relation

$${t}_{pk} = \frac{1}{{V}_{disp}} r + {t}_{0} \qquad ( 25 )$$

Where t₀ is an intercept. Similarly, differentiation of (23) with respect to time gives expression for the arrival time and group speed Vvel measured using the particle velocity signal. For XCORR measurements, the time lag τ between shear wave signals u(r₁,θ,t) and u(r₂,θ,t) observed at positions r1 and r2 can be found by calculating the cross-correlation [symbol] (r) between these signals,

[symbol]

(τ)=∫₋ₓ^{x}u(r₁,θ,t)u(r₂,θ,t+τ)dt. (26)

This expression can be evaluated using the cross correlation theorem [23] which expresses the Fourier transform of e [symbol] (τ) as the product of the Fourier domain signals ũ*(τ₁,θ,ω) and ũ(τ₂,θ,ω) given (16) and (20). Then, calculating the inverse Fourier transform gives [symbol] (τ).

$$( r ) = \frac{1}{2 π} {\int }_{- ∞}^{∞} {\tilde{u}}^{*} ( {r}_{1} , θ , ω ) \tilde{u} ( {r}_{2} , θ , ω ) {e}^{i ω τ} d ω . \qquad ( 27 )$$

The maximum cross-correlation can be found by setting the derivative d [symbol] (τ)/dτ to zero,

$$\frac{d}{dr} ( r ) = 0 = {\int }_{- ∞}^{∞} {\tilde{u}}^{*} ( {r}_{1} , θ , ω ) \tilde{u} ( {r}_{2} , θ , ω ) ω {e}^{i ω {τ}_{max}} d ω \qquad ( 28 )$$

where τₘₐₓ indicates the lag corresponding to the maximum cross-correlation. Then the group velocity is given by

$${V}_{disp} = \frac{{r}_{2} - {r}_{1}}{{τ}_{max}} \qquad ( 29 )$$

and typically, the group speed is estimated using a linear regression of time lag as a function of position. A similar expression for the group speed Vᵥₑₗ can be found using the particle velocity signals v(τ,θ,t).

It may be determined that the arrival times (24) and (28) using the TTP and XCORR estimators are not equivalent, and in addition, these expressions do not give perfectly linear relations between the arrival time and position. Thus, a unique, analytic expression for the group shear wave speeds Vdisp and Vvel may not be obtainable. Instead these speeds are determined by analyzing the shear wave signals using the same arrival time estimator and linear regression range as used for the analysis of experimentally measured signals. This analysis can be performed using the explicit relations for tpk in (24) and τmax in (28), or simply by finding the TTP or maximum XCORR signal using the shear wave signals (22) and (23).

Finite element models of ARFI excitation and shear wave propagation [24] were used to validate the analytic model of shear wave propagation described in the previous section. These models simulated the three-dimensional response of a solid using LS-DYNA3D (Livermore Software Technology Corp., Livermore, Calif.) with the *MAT_VISCOELASTIC material model that describes the shear relaxation behavior as

G(t)=G∞+(G0−G∞)e−βt (30)

where G0 and G∞ are short-time and long-time (infinite) shear moduli, respectively, and β is the decay constant. This material model is equivalent to a Standard Linear model of viscoelasticity [7] represented by a spring with stiffness μ1=G∞ in parallel with a series combination of spring with stiffness μ2=G0−G∞ and dashpot with viscosity η=(G0−G∞)/β. For this model, the shear modulus is given by [8]

$$μ ( ω ) = \frac{{μ}_{1} {μ}_{2} + i ω η ( {μ}_{1} + {μ}_{2} )}{{μ}_{2} + i ω η} . \qquad ( 31 )$$

This relation reduces to the shear modulus (8) for a Voigt model in the limit μ2→∞ so that, in this limit, the Voigt stiffness μ0 and viscosity η are given by the LS-DYNA3D model parameters in (30) as μ0=G∞ and η=(G0−G∞)/β. Simulations were performed for 18 Voigt material models using the values of stiffness μ0 and viscosity η listed in Table 1. These values were chosen to correspond to the ranges of stiffness and viscosity measured in human liver [25]. To use the LS-DYNA3D material model (30), the parameter G0 was set equal to a constant value of 5 MPa for all material property permutations, and G∞ and β were calculated to give the required values of the Voigt model parameters μ0 and η. Poisson's ratio was set to v=0.4999. Comparing the shear moduli calculated using the 3-parameter modulus (31) and the Voigt modulus (8) indicated that the RMS difference between these moduli was less than 0.3% of the moduli over a frequency range of 0-2000 Hz for all of the material models considered.

The finite element mesh was rectilinear, using cubic, hexahedral elements with 0.25 mm node spacing, extending 2.5×5.0×12.0 cm3 in the elevation, lateral and axial dimensions, respectively, under quarter-symmetry boundary conditions. Non-reflecting boundaries were used on the mesh faces extending away from the excitation in the elevation and lateral dimensions, along with the “top” and “bottom” faces. Default LS-DYNA hourglass control, with single-point quadrature, linear element formulations were solved for during explicit solver execution. Simulations were run for a total time of 30 ms with intermediate results saved at intervals of 0.1 ms. Both the axial and radial components of the displacement signals were saved.

The excitation force was modeled as a cylindrically symmetric Gaussian described by the relation

\tilde{f}(τ)=Aϵ^{-(r} ^{²}^{/α}^{²}⁾e^{-(z-ω)}^{²}^{/σ}^{_{z}}^{²}\hat{2} (32)

with σ_{z}=10σ, a focal depth z₀=60 mm, and amplitude A chosen empirically to an on-axis displacement of roughly 20 μm. The excitation width was set equal to σ=FWHM/2\sqrt{In 2} with FWHM=1.4 mm and was applied for a duration of 180 μs to correspond to the width and duration used in experimental measurements in human liver [26].

Calculations were performed on a Linux cluster with an average CPU speed of 2.6 GHz. In addition to LS-DYNA3D, calculations were performed using Field II [27] (below), and Matlab (The MathWorks, Natick, Mass.).

Group shear wave speeds measurements were made using a Verasonics™ Vantage Scanner and Philips™ C5-2 curvilinear array transducer. The scanner was programmed to acquire a diverging wave transmit-receive reference signal followed by the application of a focused ARFI excitation, and then followed by a series of 100 diverging wave transmit-receive tracking signals with a pulse repetition frequency of 5 kHz. The excitation force consisted of a 2.36 MHz, 400 μs pulse with lateral focus at a depth of 50 mm. Two excitation configurations were used with lateral F-numbers of F/1.5 and F/3.0. Beamformed IQ data were saved for the reference and tracking acquisitions. Displacement calculations were performed off-line between the reference and track signals using the Loupas [28] algorithm with a 1.5λ kernel.

Data was collected in four phantoms (E2297-A3, E2297-B1, E2297-C3, and E1787-1) manufactured by CIRS, Inc. (Norfolk, Va.). The first three of these phantoms are similar to viscoelastic phantoms used in the QIBA Phase-II study [29], and the last of these phantoms is approximately elastic and is one of the phantoms used in the QIBA Phase-I study [30]. Eight acquisitions were performed in each phantom to give a total of 16 left- and right-going shear wave signals. The phantom was repositioned between acquisitions to give independent speckle realizations.

Simulation data were analyzed by selecting displacement data u(r, t) at an axial position located at a depth equal to the center of excitation force. Velocity data v(r, t) were calculated by differentiation in time using finite differences. Both signals were truncated to a maximum time of 15 ms to simulate the extent of typical experimental signals and then filtered using a 50-1000 Hz band pass filter as typically used with experimental signals. Shear wave arrival times were estimated using both the TTP and XCORR estimators at lateral positions of 3-10 mm.

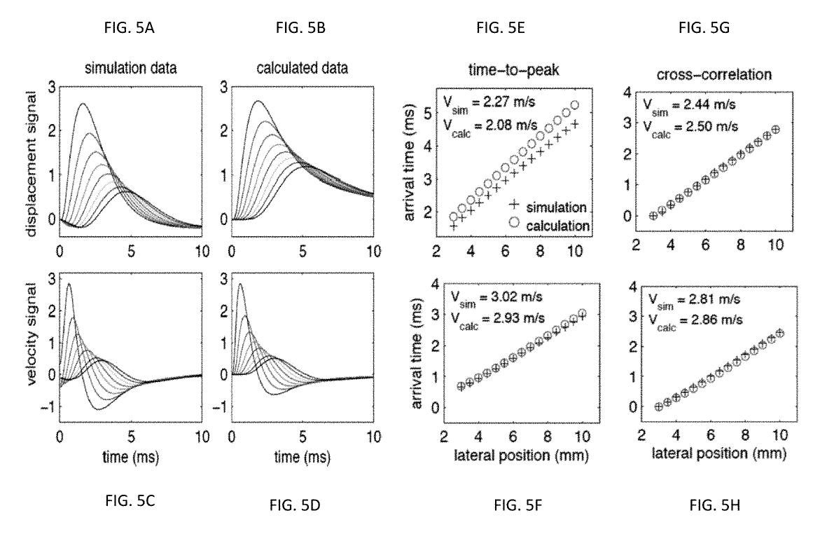

Quadratic sub-sample interpolation was used to refine the estimates of arrival time. The estimates of arrival time using XCORR were performed using a fixed reference signal located at a lateral position of 3 mm. In addition to the analysis of the axial component of displacement and velocity signals, the simulation data was also analyzed by calculating the curl of the signals. For the cylindrically symmetric excitation used with the simulations, the signals are independent of the angular coordinate, and the curl of the displacement signal is given by

$$\vec{∇} \times \vec{u} ( \vec{r} , t ) = ( \frac{\partial {u}_{r}}{\partial z} - \frac{\partial {u}_{z}}{\partial r} ) \hat{θ} \qquad ( 33 )$$

where uz and ur are the axial and radial components of the displacement signal u(\vec{τ},t). A similar expression holds for the curl of the velocity signal v(\vec{y},t). These curl signals were calculated by numerical differentiation using both the axial and radial components of the simulation data. Phantom data were processed in very much the same way as for the processing of simulation data described above with three differences. First, to reduce noise in the experimental measurements, displacement data u(\vec{τ},t) were calculated by averaging displacement data over an axial range of 10 mm centered at the focal depth of the excitation. Second, because of reverberation of the acoustic radiation from the excitation, the first tracking transmit following the excitation was discarded from the tracking signals used in the analysis of the shear wave speeds. And finally, because of the shape of the excitation force, see FIG. 5, the lateral range used for the determination of the group shear wave speeds was set to a range of 4-15 mm. In addition, group shear wave speeds in phantom data were measured using only the TTP arrival time estimator.

The calculation of the group shear wave speeds Vdisp and Vvel for specific values of stiffness and viscosity was performed by calculating the Fourier transform displacement signal ũ(r,θ,ω) at discrete frequencies in the range −8000 Hz≤f≤8000 Hz with a sample spacing of 1 Hz using (22). The shear wave displacement signal u(r, θ, t) was evaluated numerically using a discrete, inverse Fourier transform, and the shear wave velocity signal v(r, θ, t) was calculated using the finite differences of the displacement signal at sequential time steps. The arrival times and group speeds were calculated in exactly the same way as described above for the simulation and phantom data. Only shear wave propagation along the the x-axis with θ=0 in (22) was considered. The curl of the calculated signal was also found for comparison with the curl of the simulated data. In this case, however, the excitation force (12) and equation of motion (15) only allow an axial component of the displacement, and the curl of the calculated signals is given by

$$\vec{∇} \times \vec{u} ( \vec{r} , t ) = \frac{\partial {u}_{z}}{\partial r} \hat{θ} \qquad ( 34 )$$

A similar expression holds for the curl of the velocity signal v(\vec{r},t). These expressions were evaluated by numerical differentiation of the calculated signals. For the cylindrically symmetric Gaussian source used with the finite element simulations, only the m=0 term contributes in the Fourier expansion (13), and the source integral (21) was evaluated by extending the upper limit to infinity and using 10.43.23 of [31] or 6.631.4, 8.406.3, and 8.476.1 from [20],

$${S}_{0} ( ω ) = \frac{A {σ}^{2}}{2} {e}^{- {n}^{2} {σ}^{2} / 4} . \qquad ( 35 )$$

For the excitation forces used with the experimental acquisitions, Field-II [27] was used to calculate the acoustic intensity throughout a 3-dimensional volume using the known transducer element geometry with the F/1.5 and F/3.0 excitation configurations and 50 mm focal depth used in the measurements. The intensity was averaged over a 10 mm axial depth-of-field centered at the focal depth to agree with the averaging of the experimental signals over the same depth-of-field. FIG. 1 shows the two dimensional force distribution for the cases with lateral F-numbers F/1.5 (top, left) and F/3.0 (bottom, left). The infinite sum over the Fourier expansion of the shear wave displacement signal (22) was approximated by using a finite number of terms with the index m in the range −mmax≤m≤mmax. The coefficients fm(rs) in (14) were evaluated by performing the integration numerically, and the reconstructed force was calculated using the truncated sum in (13). Varying mmax indicated that the RMS differences between the original (FIG. 1, left column) and reconstructed forces are less than 0.04% for the F/1.5 case, and less than 0.02% for the F/3.0 case with mmax=10. The right column in FIG. 1 shows the reconstructed force obtained using mmax=10.

Phantom stiffness and viscosity were measured by constructing lookup tables μ0(Vdisp, Vvel) and η(Vdisp, Vvel), and using the measured group shear wave speeds Vdisp and Vvel to determine μ0 and η. The lookup tables were constructed by analytically calculating the group shear wave speeds Vdisp(μ0, η) and Vvel(μ0, η) as described above for a wide range of material stiffnesses and viscosities, and then inverting these relations.

FIGS. 5A-H illustrate graphical comparison results of shear wave propagation according to embodiments of the present disclosure. In greater detail, FIGS. 5A-5D show a comparison of results from finite element simulation data and calculated data for the case of a cylindrically symmetric Gaussian excitation with FWHM=1.4 mm and a viscoelastic material with stiffness μ0=6.0 kPa and viscosity η=3.5 Pa-s. FIGS. 5E-5H show results using the shear wave displacement and velocity signals, u(r, t) and v(r, t), respectively. The first and second columns of each Figure illustrate shear wave signals at lateral positions 3≤r≤10 mm, and the third and fourth rows of each figure illustrate arrival times as a function of lateral position calculated using the TTP and XCORR estimators. Group shear wave speeds for each case are indicated on the figure. As further shown in FIGS. 5A-5H the results shown for the simulation and calculated data are similar, but also exhibit differences in the shapes of the signals and the speeds determined from these signals. Some of the important differences between the simulation and calculated data include the following. First, the simulations were performed using a finite sized mesh while the solution of the Navier equation of motion assumes the shear wave propagation occurs in an infinite medium. One consequence of the finite mesh size is the slight decrease of the displacement signal at large lateral positions before the shear wave arrives at those positions. This decrease is due to the bulk motion of the mesh in response to the applied excitation force. Second, a fundamental difference between the simulation and calculated results is that the simulations were performed with the Poisson ratio set to 0.4999 while the calculated data were given by solutions of the Navier equation of motion (15) which assumes the shear waves propagation in an incompressible material. Third, the excitation force function (12) used in the simulations varies with axial position while the equation of motion assumes the excitation force has no axial dependence. And finally, the simulations are limited due to the finite sized node spacing and the accuracy of the non-reflecting boundary conditions.

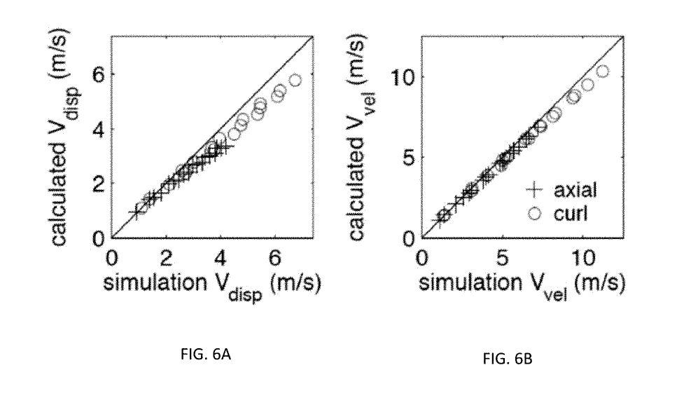

FIGS. 6A and 6B are graphs showing shear wave propagation. Referring to FIG. 6, a comparison of the group shear wave speeds Vdisp and Vvel obtained from the simulation and calculated data using the TTP arrival time estimator for the 18 material models listed in Table 1 below is illustrated.

TABLE I
VOIGT MODEL PARAMETERS μ₀ AND η USED
IN THE 18 FINITE ELEMENT SIMULATIONS.

| μ₀ (kPa) | η (Pa-s) |
|---|---|
| 1.0 | 1.0 |
| 2.0 | 0.0 |
| 3.0 | 3.0 |
| 4.0 | 5.5 |
| 5.0 | 2.0 |
| 6.0 | 3.5 |
| 7.0 | 7.5 |
| 8.0 | 5.0 |
| 9.0 | 9.5 |
| 10.0 | 4.0 |
| 11.0 | 8.0 |
| 12.0 | 11.0 |
| 13.0 | 6.0 |
| 14.0 | 9.0 |
| 15.0 | 12.0 |
| 16.0 | 7.0 |
| 17.0 | 10.5 |
| 18.0 | 13.0 |

As further shown in FIG. 6, a comparison of the group shear wave speeds calculated from the curl of the axial displacement and velocity signals using (33) and (34) is illustrated. A comparison of group shear wave speeds Vdisp (left) and Vvel (right) determined from simulation data (x-axes) and calculated data (y-axes) for the 18 materials listed in Table 1. Results are shown for the shear wave speeds determined using axial displacement and velocity data (crosses) and curl signals (circles) calculated using (33) and (34). The diagonal black lines indicate equality. The average ratios R=(V_{calc}/Vₛᵢₘ) are R_{exted}=0.88±0.06 and Rₑₓₜ=0.00±0.05 for the displacement speeds Vdisp, and Raxial=0.96±0.02 and Rₑₓₜ=0.00±0.03 for the velocity speeds Vvel indicating good agreement and a negligible impact of the dilatational components to this approach. By calculating the curl of the signals, the rotational part of the shear wave signal is isolated, and the resulting shear wave speeds are independent of the material compressibility. Thus, effects due to the use of a Poisson ratio of 0.4999 in the simulations are eliminated. The speeds from both the original and curl signals are in relatively good agreement, but that there is a systematic difference between the results with the Vcalc<Vsim for most materials.

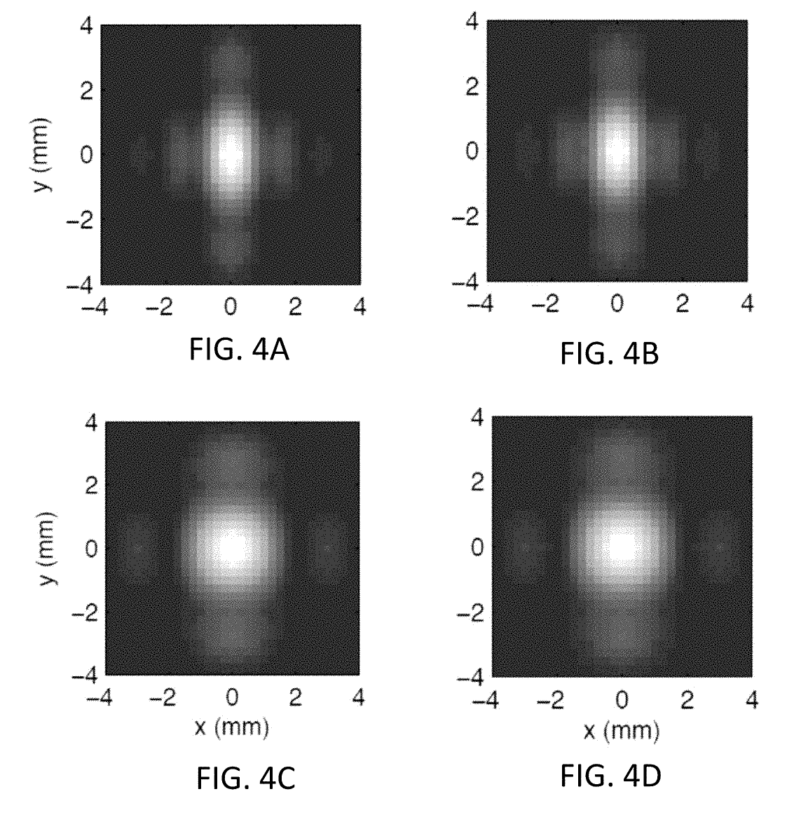

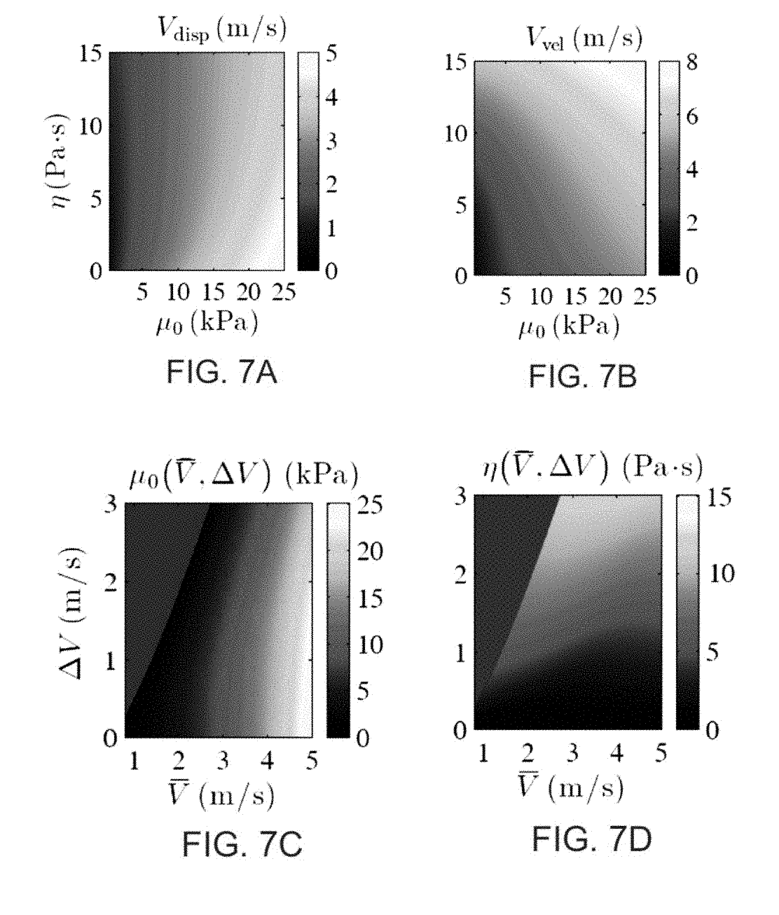

FIGS. 7A-7D depict graphical comparison results of shear wave propagation using lookup tables according to embodiments of the present disclosure. For instance, FIGS. 7A-7D illustrate the group shear wave speeds V_{disp}(μs, η) and Vᵥₑₗ (μ_{g}, η) calculated using the procedure described in Section D for a wide range of material stiffnesses μ0 and viscosities η. These calculations used the F/1.5 excitation configuration and modeled the source using the truncated Fourier expansion in (13) and (22) with −10≤m≤10 as shown in the top, right panel of FIGS. 4A-4D. The TTP estimator was used to determine the shear wave arrival time, and the linear regression was performed over a lateral range 4 mm≤r≤15 mm. Also shown in the bottom row of FIGS. 7A-7D are the lookup tables μ0 (\tilde{v}, ΔV) and η(\tilde{v}, ΔV) calculated by inverting the functional relations shown in the top row of the figure. These results have been plotted as functions of the average group speed V⁻=(Vdisp+Vvel)/2 and difference speed ΔV=Vvel−Vdisp to demonstrate the correlation between stiffness μ0 and the average group speed, and between viscosity η and the difference speed. In other words, these plots show that μ0 is a monotonic function of \tilde{v} with little dependence on ΔV, and thatη is a monotonic function of ΔV with little dependence on \tilde{v}. Thus, it was observed that average of Vdisp and Vvel is a direct measure of the material stiffness, and the difference Vvel−Vdisp is a direct measure of the material viscosity.

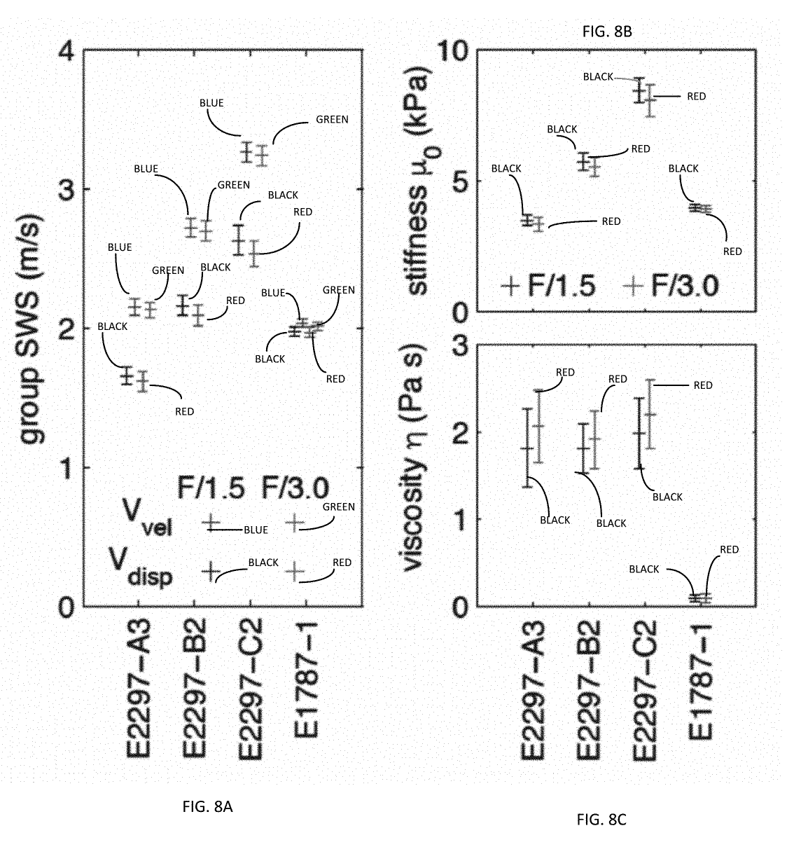

FIGS. 8A-8C depict graphical comparison results of the group shear wave speeds Vdisp and Vvel measured in four phantoms using the F/1.5 and F/3.0 excitation configurations. Referring to FIGS. 8A-8C, the group shear wave speeds Vdisp (black, red) and Vvel (blue, green) measured in four phantoms using the F/1.5 (black, blue) and F/3.0 (red, green) excitation configurations. The measured phantom stiffness (top) and viscosity (bottom) determined using the lookup tables shown in FIG. 4 for the F/1.5 (black) and F/3.0 (red) excitation configurations. Each datum point shows the mean±standard deviation from 16 measurements. For each measurement, the data points give the mean±standard deviation from 16 acquisitions. The E2297-XX viscoelastic phantoms, the group speed Vvel determined using the shear wave velocity signal is greater than the group speed Vdisp are determined using the shear wave displacement signal. For the approximately elastic phantom E1797-1, the group speeds Vdisp and Vvel are approximately equal. The group speeds are measured using the F/1.5 and F/3.0 excitation configurations are approximately equal.

Still referring to FIGS. 8A-8C, for each measurement, the data points give the mean±standard deviation from 16 acquisitions. The E2297-XX viscoelastic phantoms and the group speed Vvel are determined using the shear wave velocity signal which is greater than the group speed Vdisp being determined using the shear wave displacement signal. However, for the approximately elastic phantom E1797-1, the group speeds Vdisp and Vvel are approximately equal. Further, the group speeds measured using the F/1.5 and F/3.0 excitation configurations are approximately equal. The estimated stiffnesses μ0 and viscosities η for the same phantoms are determined using the lookup tables in FIGS. 7A-7D for the F/1.5 excitation configuration, and similar tables (not shown) for the F/3.0 excitation configuration. Results are plotted as mean±standard deviation from 16 acquisitions. As expected based on the group speeds, the stiffness varies monotonically with the speed, and that the viscosity depends on the difference speed Vvel−Vdisp. In particular, for the approximately elastic phantom, the displacement and velocity group speeds are approximately equal, and the viscosity is nearly zero.

Sensitivity of lookup tables to excitation configuration the group shear wave speeds measured in phantoms using the F/1.5 and F/3.0 excitation configurations are approximately the same. The lookup tables μ0 (\tilde{V},ΔV) and η(\tilde{V},ΔV) shown for the case of the F/1.5 excitation configuration in the bottom row of FIGS. 7A-7D appear very similar to the lookup tables for the case of the F/3.0 excitation configuration (not shown). These results may be similar because the shear wave signals are observed outside of a localized source where the shape of the source might not be expected to have a significant impact. To quantify these differences, the lookup tables can be compared by calculating the RMS fractional difference between the two excitation configurations according to the relation

$$RMS ( \frac{{[ {μ}_{0} ( \overline{V} , Δ V ) ]}_{F / 1.5} - {[ {μ}_{0} ( \overline{V} , Δ V ) ]}_{F / 8.0}}{\{ {[ {μ}_{0} ( \overline{V} , Δ V ) ]}_{F / 1.5} + {[ {μ}_{0} ( \overline{V} , Δ V ) ]}_{F / 3.0} \} / 2} ) \qquad ( 36 )$$

where RMS indicates the root-mean-square calculation of the enclosed expression. This calculation gives an RMS fractional difference of 0.2% between the μg (\tilde{V},ΔV) lookup tables. A similar calculation for the η (\tilde{V},ΔV) lookup tables (omitting the points with η (\tilde{V},ΔV)=0 at ΔV=0) gives a fraction difference of 1.5%.

To study this behavior further, lookup tables similar to those shown in FIGS. 4C and 4D were calculated using an asymmetric Gaussian excitation described by the relation

f(x, y)=F₀e^{-x}^{²}^{/σw}^{²}e^{-y}^{²}^{/σy}^{²} (37)

where the widths σx and σy are related to the Gaussian FWHM values by the relation σ=FWHM/2√ln 2. The lookup tables were calculated using the same procedure as used for the experimental F/1.5 and F/3.0 excitation configurations using a truncated Fourier expansion with-10≤m≤10 and the shear wave speeds calculated over a lateral range of 4≤r≤10 mm with the arrival times determined using the TTP estimator. The lookup tables were calculated using the Gaussian widths FWHMy of 2.0 mm, 2.5 mm, and 3.0 mm, and source aspect ratios FWHMx/FWHMy in the range 0.25-1.25. These values include cylindrically symmetric cases with FWHMx/FWHMy=1 and are similar to the size of excitation sources typically used in experimental measurements, for example, as shown in FIGS. 4A-4D. Results were analyzed by comparing each set of lookup tables to reference lookup tables determined using a cylindrically symmetric Gaussian with FWHMy=3.0.

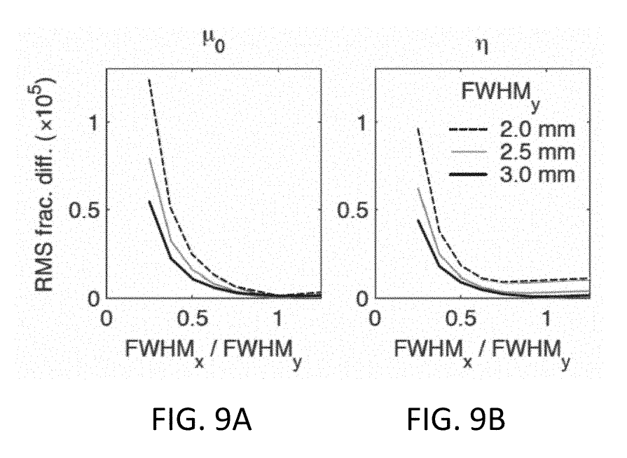

FIGS. 9A and 9B show comparison result graphics of shear wave propagation using excitation configurations. Referring to FIGS. 9A-9C, the RMS fractional difference calculated using (36). It was observed that these differences are less than 0.002%, and that the differences increase as the aspect ratio of the source deviates from unity. It is noted that the RMS fractional differences shown in FIGS. 9A and 9B for the case of an asymmetric Gaussian excitation are smaller than the differences between the F/1.5 and F/3.0 experimental configurations because of the smoother shape of the Gaussian excitation compared to the experimental excitations shown in FIGS. 4A-4D.

In other embodiments, a method is disclosed to determine the stiffness and viscosity of soft tissue by observing shear wave propagation following localized, impulsive excitations and measuring the group shear wave speeds Vdisp and Vvel of the particle displacement and particle velocity signals. The method is an improvement to existing methods that have been used to characterize the viscoelastic properties of tissue by avoiding the Fourier decomposition of shear wave signals that has typically been used to isolate individual frequency components of the signal for analysis. Thus, by avoiding the Fourier decomposition of shear wave signals, computation processing is reduced resulting more efficient processing speeds and overall efficiency of the processor of the ultrasound computing device. The present disclosure uses the measurements of group shear wave speeds which are easy to perform experimentally. Furthermore, as shown in FIGS. 9A and 9B, as long as the group speeds are measured using shear wave signals observed outside a localized source, the lookup tables μ₀(V,ΔV) and η(V,ΔV) the measured values of stiffness and viscosity, are not sensitive to the exact shape and size of the source. The results shown in FIGS. 7A-7D indicate that the difference of group shear wave speeds ΔV=Vvel−Vdisp is directly related to the viscosity of the material. Thus, this difference gives a first order measure of the material viscosity in the same way that the group shear wave speed measures the material stiffness.

One of the difficulties related to the use of at least one of the disclosed methods is that the group shear wave speeds Vdisp and Vvel do not have unique, well-defined values. For example, as shown in FIGS. 5A-5H, the TTP and XCORR arrival time estimators are not equal due to the different dependencies of the arrival times on the shear wave frequency content in (24) and (28). Thus, these estimators give different values of the group shear wave speeds. Also, the shear wave arrival time is not a linear function of position, and an analytic expression for arrival time vs. position cannot be found. Instead, the analytic group shear wave speeds must be determined by analyzing the calculated shear wave signals using the same procedure as used for the analysis of experimentally measured signals. A key assumption used in the calculation of the group shear wave speeds is that the excitation.

An example advantage in using group shear wave speeds is that properties of the excitation, including the effects of excitation duration and source size, are taken into account in the calculation of group speeds and lookup tables such as those shown in FIGS. 7A-7D. This approach is different than used in previous analysis methodologies such as the 2DFT approach which do not account for shear wave frequency content. Thus, these approaches must determine an appropriate frequency range for the analysis of experimental data and report this range as part of the result force has no dependence on the axial position z and can be described by a function that depends only on the radial coordinate r and angular coordinate θ as in (12). For more realistic cases where the excitation force varies at axial positions greater than a characteristic distance Δz, the calculated shear wave signals are expected to be accurate at radial distances r≤Δz. For the Gaussian and ARFI excitation forces used in this study, the shear waves signals were observed at positions that meet this criterion. However, other sources are more complicated. For example, an ARFI excitation with separated lateral and elevational foci can include off-axis sources [15] with a source size R that can be larger than the axial scale Δz. Shear wave signals generated by these sources will not meet the r≤Δz criterion, and either the analytic model needs to be expanded to account for the axial variation of the excitation force, or shear wave signals must be observed at positions inside the source using the full solution (19) of the equation of motion.

In an alternate embodiment, the viscoelastic properties of the material can be described using a Voigt material model. This model was chosen because it is commonly used for the analysis of viscoelastic properties of soft tissue [11, 16, 25] and requires only two parameters, stiffness and viscosity, which can be determined from two experimental measurements such as the displacement and velocity group shear wave speeds Vdisp and Vvel. Furthermore, unlike the linear dispersion model (9), the Voigt model is conveniently described by the shear modulus (8) which appears directly as a factor in the equation of motion (15). Thus, the calculated shear wave signals (22) and (23) are explicitly expressed in terms of the shear modulus through the complex wavenumber κ from (2). Extension to the case of a more complicated material model could be done using a model in which the frequency-dependent shear modulus is known, for example, the three-parameter, Standard Linear model. Such an extension would require additional experimental measurements such as the group speed Vacc measured using the shear wave acceleration signal. Even if the Voigt model is used, including the acceleration group speed would give an extra degree of freedom to allow the user to judge the appropriateness of that model. However, the calculation of the acceleration signal would require numerical differentiation of the velocity signal which would give increased noise in the acceleration signal and greater difficulty in the estimation of three material parameters from three experimental measurements.

As stated previously in alternate embodiments discussed above, the present disclosure determines the stiffness μ0 and viscosity η of soft tissue by observing shear wave propagation following localized, impulsive excitations and measuring the group shear wave speeds Vdisp and Vvel of the shear wave displacement and velocity signals. These speeds are different in viscoelastic materials with Vvel>Vdisp because the phase velocity increases with frequency and, in the frequency domain, the larger speeds at higher frequencies are weighted more heavily for the velocity signal compared to the displacement signal. For elastic materials, Vvel=Vdisp. Thus, the difference ΔV=Vvel−Vdisp gives a first order measure of the material viscosity in the same way that the group speed gives a measure of the material stiffness. Values of stiffness and viscosity are determined from the group speeds by assuming a Voigt model and solving the Navier equation of motion to determine the shear wave displacement and velocity signals which can be analyzed using time-of-flight methods. However, the shear wave arrival time is found to depend on the specific method used and is different for time-to-peak and cross-correlation estimators. Thus, the analytically calculated group shear wave speeds are determined using exactly the same procedure as used for the analysis of experimental signals. Calculating these speeds for a wide range of material stiffnesses and viscosities gives lookup tables μ0(Vdisp, Vvel) and η(Vdisp, Vvel) that can be used with the experimental group speeds. Results demonstrate that the group shear wave speeds obtained using simulation and calculated data are in agreement with minor residual differences ({tilde under (<)}12%) due to the shapes of the excitation sources and size of the modeled volumes. Results are also presented for measurements in viscoelastic phantoms and demonstrate that the analytic descriptions of shear wave signals are valid only outside of a localized source. Compared to previous methods that use Fourier decomposition of the shear wave signal to characterize the viscoelastic properties of materials, the method described here is robust due to the use of group shear wave speeds that are easily measured experimentally and the observation that the calculated lookup tables are relatively insensitive to the size and shape of the excitation force.

The disclosed methods and systems measure group shear wave speeds determined using signals such as the tissue displacement and tissue velocity signals, both of which can be measured robustly. These values can then be used to determine the stiffness and viscosity of tissue using a simple lookup table. This is made possible by the development of a method to determine the required lookup table analytically based on characteristics of the shear wave excitation force and tracking configuration, and the material properties.

In accordance with embodiments, lookup tables may be constructed by using the results of finite element models to simulate the excitation and shear wave propagation in a viscoelastic material. The tables may also be determined using the measurements of group shear wave speeds performed in calibrated phantoms with known stiffness and viscosity.

It is noted that in examples provides herein, materials are characterized in terms of stiffness and viscosity. Materials may also be characterized in other ways such as by use of group speeds using shear wave acceleration signals or signals calculated using fractional derivatives. With this data, the materials may be characterized using higher order models using, for example, three or more parameters.

It is noted that references to solving the equation of motion using the Navier equation “expressed in the temporal Fourier domain” and “using the inverse Fourier transform” seem to be very specific. The specific methods used are not critical to the goal of measuring the stiffness and viscosity using group shear wave speeds, and give only one example of how the lookup tables could be calculated.

It is also noted that the techniques described herein for determining lookup table data may alternatively be determined using any other suitable technique. For example, a lookup table may be determined using finite element simulations. In other examples, the lookup table may be determined using a finite element method, a Green's function method, one or more calibrated phantoms, or the like. The lookup table may also be determined using any other suitable technique.

The disclosed methods and systems differ from previous efforts to characterize the viscoelastic properties of tissue since it does not rely on a Fourier decomposition of the observed shear wave signal and the analysis of a frequency dependent phase velocity.

One skilled in the art will readily appreciate that the present subject matter is well adapted to carry out the objects and obtain the ends and advantages mentioned, as well as those inherent therein. The present examples along with the methods described herein are presently representative of various embodiments, are exemplary, and are not intended as limitations on the scope of the present subject matter. Changes therein and other uses will occur to those skilled in the art which are encompassed within the spirit of the present subject matter as defined by the scope of the claims.

1. A method comprising:
using an ultrasound probe to acquire soft tissue data;
determining a first group shear wave speed having a first frequency spectra;
determining a second group shear wave speed having a second frequency spectra, wherein the second frequency spectra is different than the first frequency spectra; and
determining one or more viscoelastic properties of the soft tissue based on the first group shear wave speed and the second group shear wave speed.

2. The method of claim 1, wherein determining one or more viscoelastic properties comprises determining a stiffness of the soft tissue based on the first group shear wave speed and the second group shear wave speed.

3. The method of claim 1, wherein determining one or more viscoelastic properties comprises determining a viscosity of the soft tissue based on the first group shear wave speed and the second group shear wave speed.

4. The method of claim 1, wherein determining the first group shear wave speed comprises determining the shear wave speed based on tissue displacement signals acquired by the ultrasound probe.

5. The method of claim 1, wherein determining the second group shear wave speed comprises determining the group shear wave speed based on tissue velocity signals acquired by the ultrasound probe.

6. The method of claim 1, wherein determining the group shear wave speeds comprises determining the group shear wave speeds based on fractional derivatives of the tissue motion signals acquired by the ultrasound probe.

7. The method of claim 1, further comprising using a lookup table that associates the one or more viscoelastic properties with the first group shear wave speed and the second group shear wave speed.

8. The method of claim 7, wherein the lookup table is determined using the shear wave excitation geometry, tracking configuration, and material properties of the soft tissue.

9. The method of claim 7, wherein the lookup table is determined mathematically using one of an analytic model, a finite element method model, or a Green's function method, or by solving a motion equation in the frequency domain to determine the relationship between the viscoelastic material properties and the group shear wave speeds.

10. The method of claim 7, wherein the lookup table is determined experimentally using calibrated viscoelastic phantoms.

11. The method of claim 1, further comprising presenting the one or more viscoelastic properties of the soft tissue to a user.

12. The method of claim 1, further comprising determining one or more other group shear wave speeds having one or more other frequency spectra, wherein the one or more frequency spectra are different than the first frequency spectra and the second frequency spectra, and
wherein determining the one or more viscoelastic properties of the soft tissue comprises determining the one or more viscoelastic properties of the soft tissue based on the one or more other frequency spectra.

13. The method of claim 12, further comprising using a lookup table that associates the one or more viscoelastic properties with two or more group shear wave speeds.

14. The method of claim 13, wherein the lookup table is determined using the shear wave excitation geometry, tracking configuration, and material properties of the soft tissue.

15. The method of claim 13, wherein the lookup table is determined mathematically using one of an analytic model, a finite element method model, or a Green's function method, or by solving a motion equation in the frequency domain to determine the relationship between the viscoelastic material properties and the group shear wave speeds.

16. The method of claim 13, wherein the lookup table is determined experimentally using calibrated viscoelastic phantoms.

17. A system comprising:
an ultrasound probe configured to acquire soft tissue data; and
a tissue property analyzer including at least one processor and memory configured to:
determine a first group shear wave speed having a first frequency spectra;
determine a second group shear wave speed having a second frequency spectra, wherein the second frequency spectra is different than the first frequency spectra; and
determine one or more viscoelastic properties of the soft tissue based on the first group shear wave speed and the second group shear wave speed.

18. The system of claim 17, wherein the tissue property analyzer is configured to determine a stiffness of the soft tissue based on the first group shear wave speed and the second group shear wave speed.

19. The system of claim 17, wherein the tissue property analyzer is configured to determine a viscosity of the soft tissue based on the first group shear wave speed and the second group shear wave speed.

20. The system of claim 17, wherein the tissue property analyzer is configured to determine the shear wave speed based on tissue displacement signals acquired by the ultrasound probe.

21. The system of claim 17, wherein the tissue property analyzer is configured to determine the group shear wave speed based on tissue velocity signals acquired by the ultrasound probe.

22. The system of claim 17, wherein the tissue property analyzer is configured to determine the group shear wave speeds based on fractional derivatives of the tissue motion signals acquired by the ultrasound probe.

23. The system of claim 17, wherein the tissue property analyzer is configured to use a lookup table that associates the one or more viscoelastic properties with the first group shear wave speed and the second group shear wave speed.

24. The system of claim 23, wherein the lookup table is determined using the shear wave excitation geometry, tracking configuration, and material properties of the soft tissue.

25. The system of claim 23, wherein the lookup table is determined mathematically using one of an analytic model, a finite element method model, or a Green's function method, or by solving a motion equation in the frequency domain to determine the relationship between the viscoelastic material properties and the group shear wave speeds.

26. The system of claim 23, wherein the lookup table is determined experimentally using calibrated viscoelastic phantoms.

27. The system of claim 17, wherein the tissue property analyzer is configured to present the one or more viscoelastic properties of the soft tissue to a user.

28. The system of claim 17, wherein the tissue property analyzer is configured to:
determine one or more other group shear wave speeds having one or more other frequency spectra, wherein the one or more frequency spectra are different than the first frequency spectra and the second frequency spectra; and
determine the one or more viscoelastic properties of the soft tissue based on the one or more other frequency spectra.

29. The system of claim 28, wherein the tissue property analyzer is configured to use a lookup table that associates the one or more viscoelastic properties with two or more group shear wave speeds.

30. The system of claim 28, wherein the lookup table is determined using the shear wave excitation geometry, tracking configuration, and material properties of the soft tissue.

31. The system of claim 28, wherein the lookup table is determined mathematically using one of an analytic model, a finite element method model, or a Green's function method, or by solving a motion equation in the frequency domain to determine the relationship between the viscoelastic material properties and the group shear wave speeds.

32. The system of claim 28, wherein the lookup table is determined experimentally using calibrated viscoelastic phantoms.

33. A non-transitory computer-readable medium with machine readable instructions that when executed result in a method implemented by at least one processor, the non-transitory computer-readable medium comprising:
acquiring, by the processor, soft tissue data;
determining, by the processor, a first group shear wave speed having a first frequency spectra;
determining, by the processor, a second group shear wave speed having a second frequency spectra, wherein the second frequency spectra is different than the first frequency spectra; and
determining, by the processor, one or more viscoelastic properties of the soft tissue based on the first group shear wave speed and the second group shear wave speed.
